(19) United States
(12) Patent Application Publication
(10) Pub. No.: US 2016/0175228 A1
(43) Pub. Date: Jun. 23, 2016

(54) A CREAM-LIKE SOLID AGAROSE-IN-WATER GEL PARTICLES SUSPENSION AS AN INTERMEDIATE COSMETICS PRODUCT

(71) Applicant: RED GOLD AGAROSE CORP. (PANAMA INCORPORATED), Bethenia, Panama City (PA)

(72) Inventors: Leonid LURYA, BEHOVOT (IL); Israel LEVY, MP MISGAV (IL)

(21) Appl. No.: 14/911,468

(22) PCT Filed: Aug. 12, 2014
(86) PCT No.: PCT/IB14/63878

§ 371 Date: Feb. 11, 2016

Related U.S. Application Data
(60) Provisional application No. 61/864,685, filed on Aug. 12, 2013.

(51) Int. Cl. A61K 8/73; A61Q 19/00; A61K 8/04

(52) U.S. Cl. CPC A61K 8/73; A61K 8/044; A61Q 19/00; A61K 2800/10; A61K 2800/654; A61K 2800/591; A61K 2800/48; A61K 2800/524; A61K 2800/522; A61K 2800/42; A61K 2800/412

(57) ABSTRACT

The present invention is to provide a cream-like solid agarose-in-water gel particles two-phase suspension useful for cosmetics applications, wherein the suspension is an intermediate cosmetics product. The agarose-in-water gel particles serve as a moisturizing agent, i.e. they deliver water to the skin, and impart the skin feel to the final cosmetics product. The cream-like suspension complies with ASTM skin feel analysis, which is separated in to three main evaluation sections: evaluation of the product in a petri dish, pick-up evaluation, and rub out evaluation. In the preferable embodiments the continuous phase is a water immiscible phase selected from the groups consisting of esters of the general formula RCO-OR′ and tri, di, and mono-glycerides of fatty acids. The agarose particles can be prepared from natural agarose, reduced agarose or synthetically substituted agarose.

### BACKGROUND

The cosmetics industry is a very dynamic and relentlessly evolving field with a constant quest for new natural biofriendly ingredients instead of traditionally used synthetic chemicals such as petrolatum and dimethicone. A moisturizing lotion or facial cream needs to deliver moisture to the skin and produce a smooth pleasant feeling upon application i.e. to have skin feel. The basic ingredients in creams and lotions are deionized water and lipids that can be either fats, oils or their combination. Different ratios of water/lipid content and nature of a particular lipid actually impart different skin feel to a lotion or a cream. Additional ingredients that also modify skin feel can be thickeners and humectants. Deionized water in a lotion or a cream serves as a moisturizing agent and a lipid as an occlusive agent that prevents water evaporation from the skin. Additionally cream/lotion lipids deliver water immiscible ingredients to skin.

Agarose is a nontoxic polysaccharide polymer material, generally extracted from seaweeds. Agarose is a linear polymer made up of the repeating unit of agarobiose, which is a disaccharide made up of D-galactose and 3,6-anhydro-L-galactopyranose. Agarose is available as a white powder which dissolves in near-boiling water, and forms gel when it is cooled. Agarose also can have high gel strength at low concentration, thus providing gel networks with high water content. The natural agarose polymer contains charged groups, such as pyruvate and sulphate. The agarose polysaccharide also contains uncharged methyl groups. The extent of natural methylation is directly proportional to the gelling temperature. Unexpectedly, synthetically methylated agaroses have lower, rather than higher, gelling temperatures, and the degree of synthetic methylation is inversely proportional to the melting temperature.

Commonly the natural polymer is modified in order to obtain agarose with different melting and gelling temperatures. Usually, the sulphate and pyruvate groups are reduced in order to obtain uncharged polymer. Furthermore, the charged groups can be substituted with hydroxyethyl or vinyl (allyl) groups to the various extents in order to obtain agarose with low melting temperature. There is long felt and unmet need to provide agarose based suspensions that can be used as an intermediate cosmetic product.

### SUMMARY OF THE INVENTION

It is thus one object of the present invention to provide a cream-like solid agarose-in-water gel particles suspension, comprising a continuous phase and a dispersed phase, useful for cosmetics applications, wherein said suspension is an intermediate cosmetics product.

The cream-like solid agarose particles suspension as defined above, wherein the continuous phase is a water-immiscible phase.

The cream-like solid agarose particles suspension as defined above, wherein the water-immiscible phase comprises an oil phase.

The cream-like solid agarose particles suspension as defined above, wherein the dispersed phase comprises the solid agarose-in-water gel particles.

The cream-like solid agarose particles suspension as defined above, wherein the particles are less than 100 μm in diameter.

The cream-like solid agarose particles suspension as defined above, wherein the agarose has a molecular weight of about 10,000 to 1,500,000 daltons.

The cream-like solid agarose particles suspension as defined above, wherein the agarose-in-water gel particles comprising at most 99.9% of water.

The cream-like solid agarose particles suspension as defined above, wherein the agarose is a natural unmodified polymer.

The cream-like solid agarose particles suspension as defined above, wherein the agarose acidic groups are reduced.

The cream-like solid agarose particles suspension as defined above, wherein the agarose is substituted agarose.

The cream-like solid agarose particles suspension as defined above, wherein the cream-like suspension is defined as such by ASTM E 1490-03.

The cream-like solid agarose particles suspension as defined above, wherein the cream-like suspension complies with ASTM skin feel analysis, which is separated in to three main evaluation sections: evaluation of the product in a petri dish, pick-up evaluation, and rub out evaluation.

It is another objective of the present invention to provide a method of providing a cream-like solid agarose-in-water gel particles suspension useful as an intermediate cosmetics product, comprising steps of preparing a continuous phase and a dispersed phase, useful for cosmetics applications, wherein said solid agarose-in-water gel particles forms said dispersed phase further wherein said continuous phase and said a dispersed phase comprise an intermediate cosmetics product.

The method as defined above, wherein steps of preparing said continuous phase and said dispersed phase comprising:
i. preparing a water phase by dissolving agarose powder in hot water;
ii. providing preheated water-immiscible phase;
iii. adding said water phase in small portions to said water-immiscible phase under vigorous stirring to obtain an water/oil (W/O) emulsion;
iv. cooling said emulsion down to the room temperature under slow stirring.

It is another objective of the present invention to provide a final cosmetic cream preparation, wherein said suspension is mixed with additional ingredients to produce said preparation.

The cream-like solid agarose particles suspension as defined above, wherein said suspension additionally comprises at least one ingredient selected from the group consisting of emulsifiers, emollients, thickeners, preservatives, humectants, consistency factors, antioxidants, UV blockers, vitamins, antioxidants, chelating agents, fragrance, active agents, plant extracts, coloring agents, aesthetics enhancers, vegetable oils, animal oils and fats, minerals, proteins, seaweed extracts, or any combination thereof.

The cream-like solid agarose particles suspension as defined above, wherein said ingredients further comprises at least one ingredient selected from the group consisting of natural or synthetic ambergris, allantoin, almond meal, almond oil, aloe vera, alpha beta hydroxy acid complex, alpha hydroxy acids (AHA), alpha lipoic acid, apricot kernel oil, arginine, arnica montana, ascorbic acid, avocado oil, awapuhi, azulene, babassu oil, baking soda, basil, beeswax, benzophenone-3, benzyl alcohol, beta carotene, beta glucan, birch leaf, black pepper, blue-green algae, butyrospermum parkii, calcium pantothenate, calendula, camellia sinensis (green tea), camellia sinensis (white tea), camphor, cananga ordorata flower oil, caprylic/capric triglyceride, caramel, cardamom, carica papaya, carrot oil, castor oil, castor oil phosphate, castor wax, cedarwood, centella asciatica, cetyl alcohol, chamomile, chlorella, cider vinegar, citric acid, citrus aurantium dulcis oil, citrus limonum peel, citrus medica limonum peel oil, clary sage, cocamidopropyl betaine, cocoa butter, coconut milk, coconut oil, coleus, collagen, colloidal oatmeal, coenzyme Q10, coriander, corn cob meal, cornstarch, cotton, cucumber, d-alpha tocopherol (vitamin E), dehydroxanthan gum, dihydroxyacetone, DMAE, echinacea, epsom salt, ester C, eucalyptus, evening primrose oil, fennel, flax seed oil, folic acid, frankincense, geranium maculatum oil, ginger, ginkgo biloba, ginseng, glycerin, glycolic acid, gotu kola, grape seed oil, grape skin extract, grapefruit, grapefruit (seed) extract, green tea, ground oatmeal, hibiscus, honey, honeysuckle, hyaluronic acid, hybrid safflower oil, hydrosol, hyssop, irish moss, isopropyl palmitate, jasmine oil, jojoba oil, jojoba wax, kelp, kukui nut, lactic acid, lanolinm, lavender, lavender hydrosol, lecithin, lecithin liposomes, lemon, lemon balm, lemon bioflavonoids, lemon peel, lemongrass, licorice, lime, linden flower, linum usitatissimum, macadamia nut oil, malic acid, mandarin, mangifera indica, mango butter, mentha piperita, menthol, methylparaben, mulberry leaf, myrrh, nettle, niacin—vitamin B factor, oak bark, oat kernel flour, oatmeal, octinoxate, otyl palmitate, olive oil, olive squalane, orange, orange blossom, orbignya oleifera (babassu) seed oil, orchid, palm oil, palmarosa, panthenol, papain, papaya, passion fruit, peptides, peppermint, persea gratis sima, phenoxyethanol, pine, pineapple, pomegranate, potato starch modified, powdered milk, pro-vitamin A, pro-vitamin B (biotin), pro-vitamin B5 (panthenol), pycnogenol, retinol, retinyl palmitate, rice bran oil, rose flower, rose hips, rosemary, rosemary leaf, rosemary leaf oil, safflower oil, sage, salicylic acid, saw palmetto, sea kelp, sea salt, seaweed extract, sesame oil, shea butter, sodium hyaluronate, sodium hydroxide, sorbitan stearate, sorbitol, soy bean oil, soy lecithin, soy protein, squalane, St. Johnswort, stem cells, sugar cane juice, sunflower oil, sweet almond oil, sweet orange oil, tea tree, thyme, tocopherol, tocopheryl acetate, tribehenin, turmeric, ubiquinone, ulva lactuca, vanilla, vitamin A, vitamin B complex, vitamin B factor (Inositol), vitamin B factor (Niacin B3), vitamin C (ascorbic acid), vitamin C (citric acid), vitamin C (ester C), vitamin C (L-ascorbic acid), vitamin C (magnesium ascorbyl phosphate), vitamin D, vitamin E (tocopherol), vitamin E (d-alpha tocopherol), vitamin E (tocopheryl acetate), walnut extract, walnut powder, watercress, wheat germ, wheat protein, white tea, wild thyme, wild yam root, willow bark, witch hazel, xanthan gum, ylang ylang, yucca extract, zingiber zerumbet, zinigiber officinale.

Yet another objective of the present invention is to provide a method of providing a cream-like solid agarose-in-water gel particles suspension, said method comprising steps of
i. dissolving agarose in hot water
ii. adding said dissolved agarose to a water immiscible phase thereby providing a dispersed solid agarose-in-water gel particles phase, within a continuous water immiscible phase useful for cosmetics applications,
   wherein said suspension is an intermediate cosmetics product.

The method as defined above, wherein said method comprises steps of providing said continuous phase as a water-immiscible phase.

The method as defined above, wherein said method comprises steps of providing said water-immiscible phase as an oil phase.

The method as defined above, wherein said method comprises steps of providing said dispersed phase as said solid agarose-in-water gel particles.

The method as defined above, wherein said method comprises steps of providing said particles with diameter less than 100 μm.

The method as defined above, wherein said method comprises steps of providing said agarose with a molecular weight of about 10,000 to 1,500,000 daltons.

The method as defined above, wherein said method comprises steps of providing said agarose-in-water gel particles comprising at most 99.9% wt of water.

The method as defined above, wherein said method comprises steps of providing said agarose as a natural unmodified polymer.

The method as defined above, wherein said method comprises steps of providing said agarose as agarose with reduced acidic groups.

The method as defined above, wherein said method comprises steps of providing said agarose as substituted agarose.

The method as defined above, wherein said method comprises steps of providing said cream-like suspension as is defined as such by ASTM E 1490-03.

The method as defined above, wherein said method comprises steps of providing said cream-like suspension complying with ASTM E 1490-03 skin feel analysis, which is separated into three main evaluation sections: evaluation of the product in a petri dish, pick-up evaluation, and rub out evaluation.

The method as defined above, wherein said method comprises steps of providing said suspension mixed with additional ingredients to produce said preparation.

The method as defined above, wherein said method comprises steps of providing said ingredients additionally comprising at least one ingredient selected from the group consisting of emulsifiers, emollients, thickeners, preservatives, humectants, consistency factors, antioxidants, UV blockers, vitamins, antioxidants, chelating agents, fragrance, active agents, plant extracts, coloring agents, aesthetics enhancers, vegetable oils, animal oils and fats, minerals, proteins, seaweed extracts, or any combination thereof.

The method as defined above, wherein said method comprises steps of providing said further comprising at least one ingredient selected from the group consisting of natural or synthetic ambergris, allantoin, almond meal, almond oil, aloe vera, alpha beta hydroxy acid complex, alpha hydroxy acids (AHA), alpha lipoic acid, apricot kernel oil, arginine, arnica montana, ascorbic acid, avocado oil, awapuhi, azulene, babassu oil, baking soda, basil, beeswax, benzophenone-3, benzyl alcohol, beta carotene, beta glucan, birch leaf, black pepper, blue-green algae, butyrospermum parkii, calcium pantothenate, calendula, camellia sinensis (green tea), camellia sinensis (white tea), camphor, cananga ordorata flower oil, caprylic/capric triglyceride, caramel, cardamom, carica papaya, carrot oil, castor oil, castor oil phosphate, castor wax, cedarwood, centella asciatica, cetyl alcohol, chamomile, chlorella, cider vinegar, citric acid, citrus aurantium dulcis oil, citrus limonum peel, citrus medica limonum peel oil, clary sage, cocamidopropyl betaine, cocoa butter, coconut milk, coconut oil, coleus, collagen, colloidal oatmeal, coenzyme Q10, coriander, corn cob meal, cornstarch, cotton, cucumber, d-alpha tocopherol (vitamin E), dehydroxanthan gum, dihydroxyacetone, DMAE, echinacea, epsom salt, ester C, eucalyptus, evening primrose oil, fennel, flax seed oil, folic acid, frankincense, geranium maculatum oil, ginger, ginkgo biloba, ginseng, glycerin, glycolic acid, gotu kola, grape seed oil, grape skin extract, grapefruit, grapefruit (seed) extract, green tea, ground oatmeal, hibiscus, honey, honeysuckle, hyaluronic acid, hybrid safflower oil, hydrosol, hyssop, irish moss, isopropyl palmitate, jasmine oil, jojoba oil, jojoba wax, kelp, kukui nut, lactic acid, lanolinm, lavender, lavender hydrosol, lecithin, lecithin liposomes, lemon, lemon balm, lemon bioflavonoids, lemon peel, lemongrass, licorice, lime, linden flower, linum usitatissimum, macadamia nut oil, malic acid, mandarin, mangifera indica, mango butter, mentha piperita, menthol, methylparaben, mulberry leaf, myrrh, nettle, niacin—vitamin B factor, oak bark, oat kernel flour, oatmeal, octinoxate, otyl palmitate, olive oil, olive squalane, orange, orange blossom, orbignya oleifera (babassu) seed oil, orchid, palm oil, palmarosa, panthenol, papain, papaya, passion fruit, peptides, peppermint, persea gratis sima, phenoxyethanol, pine, pineapple, pomegranate, potato starch modified, powdered milk, pro-vitamin A, pro-vitamin B (biotin), pro-vitamin B5 (panthenol), pycnogenol, retinol, retinyl palmitate, rice bran oil, rose flower, rose hips, rosemary, rosemary leaf, rosemary leaf oil, safflower oil, sage, salicylic acid, saw palmetto, sea kelp, sea salt, seaweed extract, sesame oil, shea butter, sodium hyaluronate, sodium hydroxide, sorbitan stearate, sorbitol, soy bean oil, soy lecithin, soy protein, squalane, St. Johnswort, stem cells, sugar cane juice, sunflower oil, sweet almond oil, sweet orange oil, tea tree, thyme, tocopherol, tocopheryl acetate, tribehenin, turmeric, ubiquinone, ulva lactuca, vanilla, vitamin A, vitamin B complex, vitamin B factor (Inositol), vitamin B factor (Niacin B3), vitamin C (ascorbic acid), vitamin C (citric acid), vitamin C (ester C), vitamin C (L-ascorbic acid), vitamin C (magnesium ascorbyl phosphate), vitamin D, vitamin E (tocopherol), vitamin E (d-alpha tocopherol), vitamin E (tocopheryl acetate), walnut extract, walnut powder, watercress, wheat germ, wheat protein, white tea, wild thyme, wild yam root, willow bark, witch hazel, xanthan gum, ylang ylang, yucca extract, zingiber zerumbet, zinigiber officinale.

### BRIEF FIGURE DESCRIPTION

FIG. 1 illustrates a flow chart of a method of preparation of a cream-like solid agarose-in-water gel particles two-phase suspension.

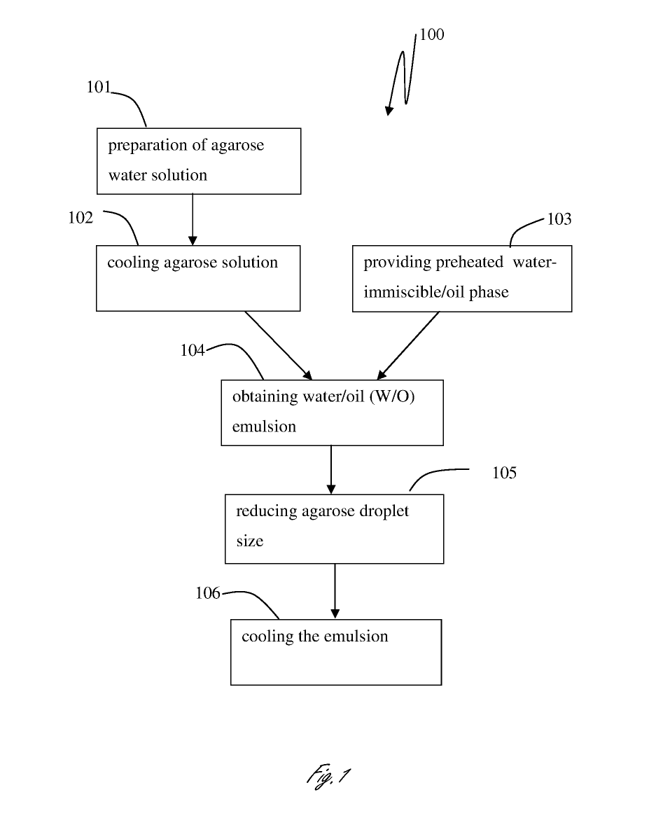

### DETAILED DESCRIPTION

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide product and method of the invention described herein.

For the purposes of the present invention, the term “cosmetic” is as defined under The Federal Food, Drug, and Cosmetic Actl sec. 201(i) as articles intended to be rubbed, poured, sprinkled, or sprayed on, introduced into, or otherwise applied to the human body for cleansing, beautifying, promoting attractiveness, or altering the appearance said cosmetic composition.

The present invention is a cream-like solid agarose-in-water gel particles two-phase suspension, which is used as an intermediate cosmetics product. The continuous phase is a water immiscible phase. The agarose-in-water particles are herein defined as gel particles consisting of agarose polymer and mainly of water, at most 99.9% wt, thus they act as a moisturizing agent and they impart skin feel to cosmetics products.

The suspension is prepared by the following exemplary method (100):
i. preparation of an agarose water solution: adding agarose powder into water and heating the water until complete dissolving of agarose (101);
ii. cooling agarose solution to 50-70° C. (102);
iii. providing a preheated up to 50-70° C. water-immiscible/oil phase (103);
iv. adding cooled agarose solution in small portions to the water-immiscible/oil phase under vigorous stirring to obtain water/oil (W/O) emulsion (104);
v. reducing the agarose droplet size (105);
vi. cooling the emulsion down to the room temperature under slow stirring (106).

More specifically, in step (i) agarose aqueous solution is provided in a preset concentration. In the preferred embodiment the agarose concentration is at least 0.1 wt %. The heating temperature depends on the agarose concentration and the agarose type. Different types of agarose can be used in various embodiments of the present invention. In different embodiments agarose has a molecular weight of about 10,000 to 1,500,000 daltons. Some embodiments of the present invention comprise natural agarose. Other embodiments of the present invention use reduced agarose. Furthermore, some other embodiments of the present invention utilize substituted agarose with low melting temperature, where substituents can be selected from the group consisting of hydroxyethyl, hydroxypropyl, methyl, allyl and acetyl substituents. For example, in one of the embodiments agarose melting temperature is 90° C. and the agarose solution is heated up to 100° C. in order to achieve complete dissolving of agarose.

In step (iv) the water phase with dissolved agarose is added to the water-immiscible phase in small portions under vigorous stirring in order to obtain an emulsion. In one embodiment the volume ratio of water phase to the water-immiscible phase is between 3:5 and 1:100, and more specifically between 1:1 and 1:100. The agarose solution forms aqueous droplets within the water-immiscible phase under vigorous stirring. In one of the embodiments of the present invention the water phase is added to the oil under vigorous stirring at 2000 rmp; this particular stirring rate produces particles with diameter of 20-50 μm. In different embodiments of the present invention the particle diameter is smaller than 100 μm.

In one of the embodiments of the present invention a thickening agent selected from the group consisting of polyethylene glycol (PEG), synthetic polymers such as carbomer, vegetable gums, various waxes, and petroleum jelly is added to the water-immiscible phase in step (iii) in order to prevent subsequent sedimentation of agarose-in-water particles in the suspension. In the particular embodiment PEG 1500 is added as a thickening agent. In an embodiment of the present invention the water-immiscible phase can be represented by the oil selected from the group consisting of natural plant oils such as sweet almond oil, wheat germ oil, and pomegranate oil. In one of the embodiments of the present invention oil phase comprises the mixture of shea butter and almond oil. In this particular embodiment the thickening agent is substituted for a co-solvent such as propylene glycol in order to dissolve shea butter in almond oil. Other embodiments of the present invention can use co-solvents such as glycerin, isododecane, and octyldodecanol and any other that is commonly used in cosmetics products. In some other embodiments an oil emulsifier can be dissolved in the oil phase. A few examples of the oil emulsifiers are the following: sorbitan sequiolate, glycerol ether polymer, polyethylene glycol hydrogenated castor oil, sorbitan trioleate, sorbitan monooleate, sorbitan tristearate or lipophilic-hydrophilic block polymers.

The oil phase in the other embodiments of the present invention can be selected from the group consisting of esters of the formula RCO-OR′ wherein R and R′ are each independently a C₁₋₂₅, preferably a C₄₋₂₀ straight or branched alkyl, alkenyl or alkoxycarbonylalkyl or alkylcarbonyloxyalkyl chains. In some other embodiments of the present invention the oil phase can be comprised of tri, di, and mono-glycerides of fatty acids.

Step (v) is an optional step provided to decrease further agarose-in-water droplet size down to 10 μm. In one embodiment the reduction of the droplet size is achieved by passing the pressurized hot emulsion (>50° C.) through a minute orifice, i.e. by using high pressure homogenizer.

In another embodiment the emulsion can be passed through a hydrophobic microporous membrane by applying high pressure. The emulsion can be transferred through the membrane at a relatively high flow rate. In some embodiments the membrane may be a glass membrane, which has been rendered hydrophobic by the chemical treatment according to the well-known methods. In another embodiment the membrane is manufactured from an inherently hydrophobic material. The term hydrophobic means that the membrane has sufficient hydrophobicity to allow passage of agarose droplets. The membrane can have any type of pore structure such as a network pores or straight cylindrical pores perpendicular to the membrane surface. The shape of the membrane can be tubular, hollow fiber, flat sheet or pleated sheet.

In step (vi) emulsion temperature must be lowered slowly at the rate below 2° C./min under slow stirring (50-200 rpm), such that agarose-in-water droplets are solidified into agarose-in-water gel particles. As mentioned above, adding PEG 1500 prevents sedimentation of the agarose-in-water gel particles.

The present invention represents solid agarose-in-water gel particles suspended in the oil phase that can be used as an intermediate cosmetics product. An example of one of the cream formulations that uses the presented cosmetics intermediate is: 9 parts of the presented suspension, 1 part of the wheat germ oil solution that includes 2% carrot seed oil, 0.2% lavender oil, 0.2% geranium oil and 0.2% patchouli oil.

The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A cream-like solid agarose-in-water gel particles suspension, comprising a continuous phase and a dispersed phase, useful for cosmetics applications, wherein said suspension is an intermediate cosmetics product.

2. The suspension of claim 1, wherein said continuous phase is a water-immiscible phase.

3. The suspension of the claim 2, wherein said water-immiscible phase comprises an oil phase.

4. The suspension of claim 1, wherein said dispersed phase comprises said solid agarose-in-water gel particles.

5. The suspension of claim 1, wherein said particles are less than 100 μm in diameter.

6. The suspension of claim 1, wherein said agarose has a molecular weight of about 10,000 to 1,500,000 daltons.

7. The suspension of claim 1, wherein said agarose-in-water gel particles comprising at most 99.9% of water.

8. The suspension of claim 6, wherein said agarose is a natural unmodified polymer.

9. The suspension of claim 6, wherein said agarose acidic groups are reduced.

10. The suspension of claim 6, wherein said agarose is substituted agarose.

11. The suspension of claim 1, wherein said cream-like suspension is defined as such by ASTM E 1490-03.

12. The suspension of claim 11, wherein said cream-like suspension complies with ASTM E 1490-03 skin feel analysis, which is separated into three main evaluation sections: evaluation of the product in a petri dish, pick-up evaluation, and rub out evaluation.

13. A method of providing a cream-like solid agarose-in-water gel particles suspension useful as an intermediate cosmetics product, comprising steps of preparing a continuous phase and a dispersed phase, useful for cosmetics applications, wherein said solid agarose-in-water gel particles forms said dispersed phase further wherein said continuous phase and said a dispersed phase comprise an intermediate cosmetics product.

14. The method of claim 13, wherein steps of preparing said continuous phase and said dispersed phase comprise:
i. preparing a water phase by dissolving agarose powder in hot water;
ii. providing preheated water-immiscible phase;
iii. adding said water phase in small portions to said water-immiscible phase under vigorous stirring to obtain an water/oil (W/O) emulsion;
iv. cooling said emulsion down to the room temperature under slow stirring.

15. A final cosmetic cream preparation, wherein said suspension is mixed with additional ingredients to produce said preparation.

16. A suspension according to claim 1, wherein said suspension additionally comprises at least one ingredient selected from the group consisting of emulsifiers, emollients, thickeners, preservatives, humectants, consistency factors, antioxidants, UV blockers, vitamins, antioxidants, chelating agents, fragrance, active agents, plant extracts, coloring agents, aesthetics enhancers, vegetable oils, animal oils and fats, minerals, proteins, seaweed extracts, or any combination thereof.

17. The suspension of claim 16, wherein said ingredients further comprises at least one ingredient selected from the group consisting of natural or synthetic ambergris, allantoin, almond meal, almond oil, aloe vera, alpha beta hydroxy acid complex, alpha hydroxy acids (AHA), alpha lipoic acid, apricot kernel oil, arginine, arnica montana, ascorbic acid, avocado oil, awapuhi, azulene, babassu oil, baking soda, basil, beeswax, benzophenone-3, benzyl alcohol, beta carotene, beta glucan, birch leaf, black pepper, blue-green algae, butyrospermum parkii, calcium pantothenate, calendula, camellia sinensis (green tea), camellia sinensis (white tea), camphor, cananga ordorata flower oil, caprylic/capric triglyceride, caramel, cardamom, carica papaya, carrot oil, castor oil, castor oil phosphate, castor wax, cedarwood, centella asciatica, cetyl alcohol, chamomile, chlorella, cider vinegar, citric acid, citrus aurantium dulcis oil, citrus limonum peel, citrus medica limonum peel oil, clary sage, cocamidopropyl betaine, cocoa butter, coconut milk, coconut oil, coleus, collagen, colloidal oatmeal, coenzyme Q10, coriander, corn cob meal, cornstarch, cotton, cucumber, d-alpha tocopherol (vitamin E), dehydroxanthan gum, dihydroxyacetone, DMAE, echinacea, epsom salt, ester C, eucalyptus, evening primrose oil, fennel, flax seed oil, folic acid, frankincense, geranium maculatum oil, ginger, ginkgo biloba, ginseng, glycerin, glycolic acid, gotu kola, grape seed oil, grape skin extract, grapefruit, grapefruit (seed) extract, green tea, ground oatmeal, hibiscus, honey, honeysuckle, hyaluronic acid, hybrid safflower oil, hydrosol, hyssop, irish moss, isopropyl palmitate, jasmine oil, jojoba oil, jojoba wax ,kelp, kukui nut, lactic acid, lanolinm, lavender, lavender hydrosol, lecithin, lecithin liposomes, lemon, lemon balm, lemon bioflavonoids, lemon peel, lemongrass, licorice, lime, linden flower, linum usitatissimum, macadamia nut oil, malic acid, mandarin, mangifera indica, mango butter, mentha piperita, menthol, methylparaben, mulberry leaf, myrrh, nettle, niacin—vitamin B factor, oak bark, oat kernel flour, oatmeal, octinoxate, otyl palmitate, olive oil, olive squalane, orange, orange blossom, orbignya oleifera (babassu) seed oil, orchid, palm oil, palmarosa, panthenol, papain, papaya, passion fruit, peptides, peppermint, persea gratissima, phenoxyethanol, pine, pineapple, pomegranate, potato starch modified, powdered milk, pro-vitamin A, pro-vitamin B (biotin), pro-vitamin B5 (panthenol), pycnogenol, retinol, retinyl palmitate, rice bran oil, rose flower, rose hips, rosemary, rosemary leaf, rosemary leaf oil, safflower oil, sage, salicylic acid, saw palmetto, sea kelp, sea salt, seaweed extract, sesame oil, shea butter, sodium hyaluronate, sodium hydroxide, sorbitan stearate, sorbitol, soy bean oil, soy lecithin, soy protein, squalane, St. Johnswort, stem cells, sugar cane juice, sunflower oil, sweet almond oil, sweet orange oil, tea tree, thyme, tocopherol, tocopheryl acetate, tribehenin, turmeric, ubiquinone, ulva lactuca, vanilla, vitamin A, vitamin B complex, vitamin B factor (Inositol), vitamin B factor (Niacin B3), vitamin C (ascorbic acid), vitamin C (citric acid), vitamin C (ester C), vitamin C (L-ascorbic acid), vitamin C (magnesium ascorbyl phosphate), vitamin D, vitamin E (tocopherol), vitamin E (d-alpha tocopherol), vitamin E (tocopheryl acetate), walnut extract, walnut powder, watercress, wheat germ, wheat protein, white tea, wild thyme, wild yam root, willow bark, witch hazel, xanthan gum, ylang ylang, yucca extract, zingiber zerumbet, zinigiber officinale.

18. A method of providing a cream-like solid agarose-in-water gel particles suspension, said method comprising steps of
i. dissolving agarose in hot water
ii. adding said dissolved agarose to a water immiscible phase thereby providing a dispersed solid agarose-in-water gel particles phase, within a continuous water immiscible phase useful for cosmetics applications,
wherein said suspension is an intermediate cosmetics product.

19. The method according to claim 18 wherein said method comprises steps of providing said continuous phase as a water-immiscible phase.

20. The method according to claim 18 wherein said method comprises steps of providing said water-immiscible phase as an oil phase.

21. The method according to claim 18 wherein said method comprises steps of providing said dispersed phase as said solid agarose-in-water gel particles.

22. The method according to claim 18 wherein said method comprises steps of providing said particles with diameter less than 100 μm.

23. The method according to claim 18 wherein said method comprises steps of providing said agarose with a molecular weight of about 10,000 to 1,500,000 daltons.

24. The method according to claim 18 wherein said method comprises steps of providing said agarose-in-water gel particles comprising at most 99.9% wt of water.

25. The method according to claim 18 wherein said method comprises steps of providing said agarose as a natural unmodified polymer.

26. The method according to claim 18 wherein said method comprises steps of providing said agarose as agarose with reduced acidic groups.

27. The method according to claim 18 wherein said method comprises steps of providing said agarose as substituted agarose.

28. The method according to claim 18 wherein said method comprises steps of providing said cream-like suspension as is defined as such by ASTM E 1490-03.

29. The method according to claim 18 wherein said method comprises steps of providing said cream-like suspension complying with ASTM E 1490-03 skin feel analysis, which is separated into three main evaluation sections: evaluation of the product in a petri dish, pick-up evaluation, and rub out evaluation.

30. The method according to claim 18 wherein said method comprises steps of providing said suspension mixed with additional ingredients to produce said preparation.

31. The method according to claim 18 wherein said method comprises steps of providing said ingredients additionally comprising at least one ingredient selected from the group consisting of emulsifiers, emollients, thickeners, preservatives, humectants, consistency factors, antioxidants, UV blockers, vitamins, antioxidants, chelating agents, fragrance, active agents, plant extracts, coloring agents, aesthetics enhancers, vegetable oils, animal oils and fats, minerals, proteins, seaweed extracts, or any combination thereof.

32. The method according to claim 18 wherein said method comprises steps of providing said further comprising at least one ingredient selected from the group consisting of natural or synthetic ambergris, allantoin, almond meal, almond oil, aloe vera, alpha beta hydroxy acid complex, alpha hydroxy acids (AHA), alpha lipoic acid, apricot kernel oil, arginine, arnica montana, ascorbic acid, avocado oil, awapuhi, azulene, babassu oil, baking soda, basil, beeswax, benzophenone-3, benzyl alcohol, beta carotene, beta glucan, birch leaf, black pepper, blue-green algae, butyrospermum parkii, calcium pantothenate, calendula, camellia sinensis (green tea), camellia sinensis (white tea), camphor, cananga ordorata flower oil, caprylic/capric triglyceride, caramel, cardamom, carica papaya, carrot oil, castor oil, castor oil phosphate, castor wax, cedarwood, centella asciatica, cetyl alcohol, chamomile, chlorella, cider vinegar, citric acid, citrus aurantium dulcis oil, citrus limonum peel, citrus medica limonum peel oil, clary sage, cocamidopropyl betaine, cocoa butter, coconut milk, coconut oil, coleus, collagen, colloidal oatmeal, coenzyme Q10, coriander, corn cob meal, cornstarch, cotton, cucumber, d-alpha tocopherol (vitamin E), dehydroxanthan gum, dihydroxyacetone, DMAE, echinacea, epsom salt, ester C, eucalyptus, evening primrose oil, fennel, flax seed oil, folic acid, frankincense, geranium maculatum oil, ginger, ginkgo biloba, ginseng, glycerin, glycolic acid, gotu kola, grape seed oil, grape skin extract, grapefruit, grapefruit (seed) extract, green tea, ground oatmeal, hibiscus, honey, honeysuckle, hyaluronic acid, hybrid safflower oil, hydrosol, hyssop, irish moss, isopropyl palmitate, jasmine oil, jojoba oil, jojoba wax, kelp, kukui nut, lactic acid, lanolinm, lavender, lavender hydrosol, lecithin, lecithin liposomes, lemon, lemon balm, lemon bioflavonoids, lemon peel, lemongrass, licorice, lime, linden flower, linum usitatissimum, macadamia nut oil, malic acid, mandarin, mangifera indica, mango butter, mentha piperita, menthol, methylparaben, mulberry leaf, myrrh, nettle, niacin—vitamin B factor, oak bark, oat kernel flour, oatmeal, octinoxate, otyl palmitate, olive oil, olive squalane, orange, orange blossom, orbignya oleifera (babassu) seed oil, orchid, palm oil, palmarosa, panthenol, papain, papaya, passion fruit, peptides, peppermint, persea gratissima, phenoxyethanol, pine, pineapple, pomegranate, potato starch modified, powdered milk, pro-vitamin A, pro-vitamin B (biotin), pro-vitamin B5 (panthenol), pycnogenol, retinol, retinyl palmitate, rice bran oil, rose flower, rose hips, rosemary, rosemary leaf, rosemary leaf oil, safflower oil, sage, salicylic acid, saw palmetto, sea kelp, sea salt, seaweed extract, sesame oil, shea butter, sodium hyaluronate, sodium hydroxide, sorbitan stearate, sorbitol, soy bean oil, soy lecithin, soy protein, squalane, St. Johnswort, stem cells, sugar cane juice, sunflower oil, sweet almond oil, sweet orange oil, tea tree, thyme, tocopherol, tocopheryl acetate, tribehenin, turmeric, ubiquinone, ulva lactuca, vanilla, vitamin A, vitamin B complex, vitamin B factor (Inositol), vitamin B factor (Niacin B3), vitamin C (ascorbic acid), vitamin C (citric acid), vitamin C (ester C), vitamin C (L-ascorbic acid), vitamin C (magnesium ascorbyl phosphate), vitamin D, vitamin E (tocopherol), vitamin E (d-alpha tocopherol), vitamin E (tocopheryl acetate), walnut extract, walnut powder, watercress, wheat germ, wheat protein, white tea, wild thyme, wild yam root, willow bark, witch hazel, xanthan gum, ylang ylang, yucca extract, zingiber zerumbet, zinigiber officinale.
